(19) United States
(12) Patent Application Publication
(10) Pub. No.: US 2024/0125760 A1
(43) Pub. Date: Apr. 18, 2024

(54) Novel Oil Extraction and Geochemical Fingerprinting Technology

(71) Applicant: Le Lu, Covina, CA (US)

(72) Inventors: Le Lu, Covina, CA (US); Yao Li, Covina, CA (US); Wanyi Zhang, Covina, CA (US); Yongchun Tang, Covina, CA (US)

(73) Assignee: GeoIsoChem Corporation, Covina, CA (US)

(21) Appl. No.: 17/865,296

(22) Filed: Jul. 14, 2022

(51) Int. Cl. G01N 33/28; E21B 49/00

(52) U.S. Cl. CPC G01N 33/2823; E21B 49/00

(57) ABSTRACT

Method of characterizing the chemical signature of petroleum oil contained in reservoir rock for the purpose of production allocation, estimation of vertical drainage, and monitoring of production. Reservoir rock samples from petroleum pay zones are cleaned from surface contaminations, and then loaded with water into pressure vessels and seals to airtight. The vessels are heat treated with elevated pressure to release trapped oil. Oil is extracted from the products from the vessels and analyzed for chemical compositions. The measured compositions of extracted oils are used to construct geochemical fingerprinting for the characterization of fluid-in-place in the pay zones.

### BACKGROUND

### Field of the Invention

This invention relates to methods for estimation of geochemical characteristics of reservoir fluids for the purpose of production allocation and reservoir monitoring. More particularly, this invention relates to extracting hydrocarbons from reservoir rocks (core, side wall core, drill cuttings, etc.) to measure fingerprinting of in-place oils in petroleum reservoirs.

### Background

Oil fingerprinting has been widely used to quantify reservoir constituents like mixing, migration, source, and production allocation. The understanding of the natural variation of fluid compositions plays the essential role in reservoir monitoring, optimizing well placement and landing, optimization of well completion and hydraulic fracturing. Fingerprinting of produced oil is well established geochemical procedure applied in many conventional oil fields worldwide. However, when the end-member oil sampling is not available, especially in unconventional resources, it is a challenge to establish oil fingerprinting from reservoir rock samples. Existing technologies include 1) thermally evaporating oils from dry rock samples, and 2) solvent extraction. The bias in hydrocarbon compositions between extracted oil and the produced oil from the same rock can induce considerable error in the rock extract fingerprinting, rendering less accurate reservoir monitoring results. Therefore, a need exists in the field for novel oil extraction and fingerprinting methods that can obtain the best analog of the produced oil from the reservoir rock samples and generating reliable geochemical fingerprinting.

### BRIEF SUMMARY OF THE INVENTION

The present invention is a novel oil extraction and fingerprinting method for production allocation and reservoir monitoring purposes. It comprises of:
Clean rock samples to remove surface contaminations that can interfere with fingerprinting.
Use a closed system heat process to the rock sample to reopen micro-pores and release trapped oils.
Extract oils by heat, solvent, or reduced surface tension.
Analyze geochemical fingerprinting of the extract.

### BRIEF DESCRIPTION OF THE DRAWINGS

FIG. 1 demonstrates an example of oil extraction using closed system heat preparation with elevated temperature and pressure.

### DETAILED DESCRIPTION OF THE INVENTION

A proposed method and analysis technique using organic geochemistry fingerprinting analysis for formation rock samples to establish key identifiers and information pertaining to petroleum resources including but not limited to production allocation, estimation of vertical drainage profile, oil mixing, horizontal characterization, and stratigraphic control indicators.

This invention involves preparing formation rock samples, including but not limited to drill cuttings, core, sidewall core, and rock chips. The rock samples are cleaned with water-based solution of low surface tension surfactants to remove any mud residuals and contaminations from the rock surface. Larger rock grains are ground under controlled temperature to 3.5 to 500 mesh size.

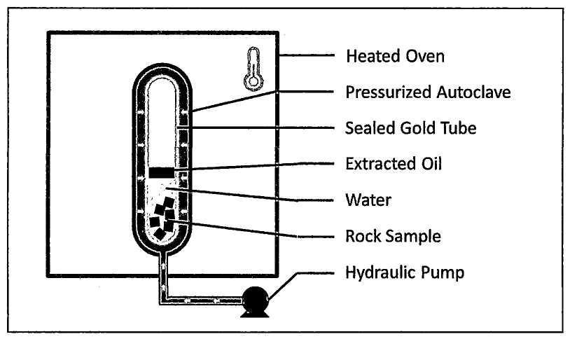

In the next step, the invention uses closed vessels to confine the rock samples during the treatment. The material of closed vessels includes, but is not limited to, gold, copper, stainless steel tubes. Flexible materials, such as gold, are generally preferred since it can be squeezed during the process by hydraulic fluids to apply external pressure onto the rock samples. The prepared rock sample is loaded, with or without added water, into a closed vessel. The vessel is then sealed to air-tight and heated isothermally at the temperature in the range of 50° C. to 350° C., preferably slightly above the sample's original reservoir temperature, for the duration of up to 2 months (FIG. 1).

The oil in the product in the closed vessels after the heating process is extracted using oil extraction methods including but not limited to: 1) thermally evaporation of hydrocarbons and collection by an analyzer; 2) solvent extraction in which organic solvents are used to dissolve and separate oils from the rest of the products; 3) imbibition, which is preferred, in which oil is displaced by water from the rocks by reduced surface tension.

The extracted oil is then analyzed to obtain high resolution geochemical fingerprinting using analysis methods including, but not limited to, gas chromatography with flame ionization detector (GC-FID), gas chromatography mass spectrometry (GC-MS), and gas chromatography with isotope ratio mass spectrometry (GC-IRMS).

1. A method of extracting crude oil from reservoir rock samples, comprising:
a) Obtaining a plurality of rock samples from a plurality of pay zones of said one or more reservoir(s).
b) Preparing said plurality of rock samples to remove contaminations.
c) Grinding large grains in said plurality of rock samples to finer particles.
d) Sealing said plurality of prepared rock samples in a plurality of air-tight vessels.
e) Heating said plurality of vessels to release trapped oil from the rock.
f) Extracting generated oil from the said plurality of vessels; and
g) Geochemically fingerprinting said extracted oil from the plurality of rock samples to provide a plurality of oil fingerprints.

2. The method of claim 1, wherein said fingerprints comprise ratios of chemical compounds that are consistent between rock samples in a) and produced oil samples in the field.

3. The method of claim 1, wherein said reservoir rock is sampled from cuttings drilled with oil-based-mud.

4. The method of claim 1, wherein said rock sample preparation step uses water to wash out contaminations.

5. The method of claim 4, wherein low surface tension surfactants are added to the said water to enhance the removal of contaminations.

6. The method of claim 1, wherein said vessels in d) are made of gold, brass, stainless steel, and other metal or non-metal materials that are inert to organic and inorganic compounds in said rock samples in the temperature range of the process in e).

7. The method of claim 1, wherein water is added in the vessels with rock samples such that rock particles are submerged in water.

8. The method of claim 1, wherein the temperature in the heating step e) is preferably slightly above the rock samples' original reservoir temperature, but not exceeding 350° C. or the equivalent cracking temperature of hydrocarbons.

9. The method of claim 1, wherein confining pressure, preferably the rock samples' original reservoir pressure, is applied to the said rock samples and water in the sealed vessels by squeezing the flexible vessel walls using hydraulics.

10. The method of claim 1, wherein the extraction step in f) uses phase separation of oil and water, solvent separation of dissolved oil in water, solvent extraction of oil adsorbed on the rock surface, or thermally evaporating then condensing the oil.

11. The method of claim 1, wherein the fingerprinting step in g) uses GC, GCxGC, MS, GC-MS, FTICR-MS, TLC, 2D TLC, CE, HPLC, FTIR Spectrophotometry, XRF, AAS, ICP-MS, IC, NMR, GCxGC-TOFMS, SARA, CHNOS analysis, elemental analysis, or GC/IR-MS.
